(19) United States
(12) Patent Application Publication
(10) Pub. No.: US 2015/0223705 A1
(43) Pub. Date: Aug. 13, 2015

(54) MULTI-FUNCTIONAL USER WEARABLE PORTABLE DEVICE

(71) Applicant: Rajendra Padma SADHU, Somerset, NJ (US)

(72) Inventor: Rajendra Padma SADHU, Somerset, NJ (US)

(21) Appl. No.: 14/682,528

(22) Filed: Apr. 9, 2015

Related U.S. Application Data
(63) Continuation-in-part of application No. 14/025,475, filed on Sep. 12, 2013.
Application No. 14/025,475 is a continuation of application No. 13/105,925, filed on May 12, 2011, now Pat. No. 8,568,313.
Application No. 13/105,925 is a continuation-in-part of application No. 12/722,554, filed on Mar. 12, 2010, now abandoned.

(51) Int. Cl. A61B 5/0205; G05B 15/02; H04L 12/28; A61B 5/00

(52) U.S. Cl. CPC A61B 5/02055; A61B 5/6803; A61B 5/681; A61B 5/6804; A61B 5/0022; A61B 5/742; A61B 5/747; A61B 5/4818; A61B 5/7455; A61B 5/7405; G05B 15/02; H04L 12/2803; A61B 5/02438

(57) ABSTRACT

A multipurpose wearable portable device is disclosed that can collect and process data/information/parameter values from one or more sensors and compares the same with one or more predefined/threshold value to suggest one or more actions and/or generate alerts/messages/suggestions to be performed by one or a combination of remote system, wearer, home automation network, healthcare provider, doctor, caretaker, among other stakeholders.

### CROSS REFERENCE OF RELATED APPLICATION

This is a Continuation-In-Part application of U.S. patent application Ser. No. 14/025,475, filed Sep. 12, 2013 which is a Continuation of U.S. patent application Ser. No. 13/105,925, filed May 12, 2011, that is now U.S. Pat. No. 8,568,313, which is a Continuation-In-Part of U.S. patent application Ser. No. 12/722,554, filed Mar. 12, 2013, the contents of each of which are hereby incorporated by reference in their entireties.

### BACKGROUND

The background description includes information that may be useful in understanding exemplary embodiments described herein. It is not an admission that any of the information provided herein is prior art or relevant to the presently claimed invention, or that any publication specifically or implicitly referenced is prior art.

In the recent past, two technology sectors, namely healthcare sector and home automation control sector have provided improved comfort and quality of life to people, giving enhanced life expectancy. Due to availability of new technologies in home automation control industry and improvement of medical technologies, diseases that were earlier deemed difficult for treatment in the past such as acute diseases and severe diseases, have gradually become treatable. There are several diseases that can be controlled and/or maintained through proper monitoring and therefore require long-term observation/treatment.

Home automation control industry, on the other hand, has provided better control of home and office premises with several sensors installed within the building to monitor for alerting whereabouts of people inside the building/room along with detecting/monitoring/controlling temperature, humidity, potential fire condition, security threats, among other defined/desired parameters, and also taking automated actions such as adjusting AC control, alerting caretakers/energy team about people inside the building or of any emergency condition. Healthcare industry has used similar concepts to collect sensory data from users, and provide healthcare services from remote places using remotely configured healthcare systems. Such services includes collecting sensory data such as body temperature, physiological parameters, psychological parameters, and biological parameters, heart-beat, pulse rate, blood pressure, blood sugar, breath rate and other parameters to continuously monitor health of user/patients and generate alerts to intimate caretakers if some parameters exceed predefined thresholds. A remote health care system can automatically inform nearby emergency medical team to reach patient's premises in case of emergency situation. Such remote healthcare services can send alerts to care provider such as a local nurse, who takes care of a patient, or send messages/alerts to a nearby doctor to attend the patient on emergency cases. Using such remote healthcare systems, patients are kept at home and their health parameters are continuously readout from one or more remote site(s) and appropriate suggestions are provided to the patient or local care provider.

A patient suffering from a chronic disease usually needs long-term mediation, and therefore needs to accurately follow the medication timely if an effective and planned treatment is desired. Other important events such as revisiting hospital for consultation, undertaking physical exercises, among other like events, on schedule, also need to be diligently followed up, which in most cases, is difficult to follow. Existing healthcare systems also provide alerts to patients to take their medicine on time and alert him/her to visit his/her doctor at scheduled times. State of the art remote healthcare systems further use a variety of healthcare devices to continuously monitor users in a non-invasive manner. However, these devices need the user to be near the medical/sensory device that is going to monitor the user, making movement of the patient constrained.

There may also be certain users who are not critically ill and do not need to be confined within the home, but need regular monitoring. Above mentioned monitoring devices typically don't work for such cases as its tedious for users to carry these devices wherever they go. In the past, patients have also been provided with a panic button that interlinks the user to an emergency response team via landline or mobile telephone. If the user is suddenly disabled during a sudden health crisis, such as in a heart attack or a serious fall situation, the panic-button type devices become useless. Furthermore, even if a user is able to press the button, he/she should be within effective wireless transmission distance to a device that dials the telephone to report the emergency. The panic button type devices therefore are not effective for the users when the users are in unconscious state or rendered incapacitated by a fall or other sudden medical condition inside or outside the home. Furthermore, no vital information on the user's status such as heart rate, blood pressure, breath rate, body temperature, oxygen level, and the like, are transmitted to the response team to provide further medical assistance. Consequently, it is difficult for the response team to diagnose and provide treatment to the user.

Few more advanced remote healthcare systems require a separate device to be placed at home or at user premises that collects healthcare data and/or home automation control data from one or more sensors and sends it to remote device/central server for analysis and alert generation. Using sensory data such as room temperature and humidity, the home automation control system can automatically control air conditioner and humidifier, whereas the healthcare system can determine health status of one or more users. In some known implementations, an in-house configured device collects data, analyzes it, and generates alerts locally and to one more remote devices/users/caretakers. Generally, these healthcare devices are not portable and therefore are restricted to one geographical area, which limits the movement of a user/patient if he/she needs to be regularly monitored.

In existing systems, even if after getting alert messages, a doctor or nurse or emergency team reaches the user premises, they may not have access to house/building and may have to either wait for a person to open the house or would have to break the door. In such emergency cases, delay of even a minute may be critical to life, and therefore existing devices are simply not sufficient for meeting the current day's requirements for fast, efficient, accurate, healthcare monitoring.

In light of the aforementioned limitations and requirements, there clearly exists a need for a user wearable portable communication device for monitoring, receiving, relaying and analyzing healthcare parameters of a user through a plurality of sensors, along with being communicatively coupled with home automation network for efficient interaction with and control of home appliances and resources.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

In accordance with teachings of the present disclosure, a multipurpose wearable portable device is disclosed, wherein the device can be configured to, by means of a plurality of sensors, collect data from various healthcare parameters, process the collected data, compare the data with one or more respective predefined values, and suggest/propose one or more actions and/or generate alerts that can be communicated to the wearer and/or to a remote device or any other stakeholder in the proposed system.

In an aspect, an object/purpose of the present disclosure is to provide a multipurpose wearable device including/coupled with a plurality of sensors that can be operatively connected with each other and configured to efficiently collect/process/monitor/alert sensory data related to personal health based parameters, and/or enable control/operation of home automation network based on predefined rules/algorithms/thresholds so as to allow communication of messages/signals/alerts to one or a combination of a wearer of the device, remote system(s), equipment's/resources, and other stakeholders such as caretakers.

An object of the present disclosure is to provide a wearable device such as a smart watch, a belt, spectacles, or a head strip that has a programmable microcontroller operatively coupled with a plurality of embedded internal/external sensors or one or more sensors connected to it through a short range wireless communication means, a processing means to process data collected from one or more sensors, an alerts generation means to generate alert for the user/caretaker/remote device, and a communication means for transmitting generated alert/message to one or more remote device or home automation control system or a remote healthcare system using an applicable communication means. One or more sensors that may be connected to the microcontroller of the wearable device can be a heart-beat sensor, a pulse rate sensor, an accelerometer, gyroscope, oxygen sensor, temperature sensor, humidity sensor, location tracking sensor, UV sensor, magnetometer, proximity sensor, among other sensors to collect desired health parameters and home automation control parameters.

Another object of the present disclosure is to provide a wearable device having a configurable one touch emergency notification bottom to notify emergency team/healthcare provider through a communication means and/or unlock the door of house/building for emergency healthcare team/health care providers. This will help the emergency rescue crew to act quickly without losing time in breaking the door.

Another object of the present disclosure is to provide an automatic system configured in a wearable device to issue command(s)/signal(s) to emergency team/healthcare provider and/or to home automation control system in case it detects an unwanted condition based on reading of one or more sensors that are coupled with the device. Based on the received command, home automation system can unlock door of house/building, adjust temperature/humidity/oxygen concentration within house/building, along with performing other actions configured to be implemented which are either preprogrammed or programmed on demand.

Yet another object of the present disclosure is to provide a user wearable portable device that performs sleep apnea test with the help of data readings from an accelerometer and a gyroscope, which can be configured in or operatively coupled with the wearable device. The sleep test, in another implementation, can further be conducted based on oxygen saturation readings from either an in-built sensor or by means of a short range wireless communication of the device with an external pulse oximeter. Results of the test can then be analyzed, shared with stakeholders including the user, doctor, caretaker, among other entities. Wearable device can alert by the wearer by either vibration or buzzer or voice and wake him up so that he will move and start breathing if the oxygen levels are below the threshold limits.

Still another object of the present disclosure is to provide a user wearable portable device that includes an embedded or operatively coupled GPS sensor(s) and/or Cellular, WiFi, ZigBee® like triangulation based location estimation system(s), which can inform a healthcare provider about actual location of the user in case of an emergency.

Still another object of the present disclosure is to provide a wearable portable device that can provide suggestions/alert to a user based on reading(s) extracted from one or more sensors, and/or adjust setting of home automation control device. For example, user wearable device of the present disclosure can retrieve temperature and humidity data from applicable sensors, and suggest the concerned user about precautions to be taken at a given temperature/humidity, and/or if authorized, automatically adjust thermostat and/or humidifier to bring temperature and humidity at the required/desired/intended level. These set of actions can be either pre-programmed or programmed on demand.

Another object of the present disclosure is to provide a wearable portable device having embedded long range wireless communication means and/or short range wireless and/or a USB interface to receive and transmit sensory data and other messages between user wearable device and a remote health care provider and/or a home automation control system.

Further object of the present disclosure is to automatically post the reading/suggestions/alert messages on a user's registered social media websites, and receive suggestions from his/her friend using a user wearable portable device. In another embodiment, any other mode of message communication such as through a phone call, SMS, PUSH message, MMS, email, among others can be incorporated.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

Various objects, features, aspects and advantages of the exemplary embodiments will become more apparent from the following detailed description of preferred embodiments, along with the accompanying drawing figures in which like numerals represent like components.

### BRIEF DESCRIPTION OF THE DRAWINGS

In the Figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label. Other objects and advantages of the present invention Will become apparent to those skilled in the art upon reading the following detailed description of the preferred embodiments, in conjunction With the accompanying drawings, Wherein like reference numerals have been used to designate like elements and Wherein:

FIG. 1 illustrates an exemplary block diagram of a user wearable portable device used for healthcare monitoring and home automation control in accordance with an embodiment of the present disclosure.

FIG. 2 illustrates an exemplary schematic view of application environment of user wearable portable device in accordance with an embodiment of the present disclosure.

FIG. 3 illustrates an exemplary block diagram of a wearable portable device having long range and short range communication capability in accordance with an embodiment of the present disclosure.

FIG. 4 illustrates an exemplary block diagram of a remote healthcare system and home automation control system using a user wearable portable device in accordance with an embodiment of the present disclosure.

FIG. 5 illustrates an exemplary block diagram of exemplary sensors embedded or attached to the user wearable portable device in accordance with an embodiment of the present disclosure.

FIG. 6 illustrates exemplary functional modules diagram of a multipurpose wearable portable device used for remote health care management and home automation control in accordance with an embodiment of the present invention.

FIG. 7 illustrates an exemplary logical table showing actions taken by the wearable device based on readings from one or more sensor in accordance with an embodiment of the present invention.

### DETAILED DESCRIPTION

Embodiments of the present invention include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware, software, firmware and/or by human operators.

Embodiments of the present invention may be provided as a computer program product, which may include a machine-readable storage medium tangibly embodying thereon instructions, which may be used to program a computer (or other electronic devices) to perform a process. The machine-readable medium may include, but is not limited to, fixed (hard) drives, magnetic tape, floppy diskettes, optical disks, compact disc read-only memories (CD-ROMs), and magneto-optical disks, semiconductor memories, such as ROMs, PROMs, random access memories (RAMs), programmable read-only memories (PROMs), erasable PROMs (EPROMs), electrically erasable PROMs (EEPROMs), flash memory, magnetic or optical cards, or other type of media/machine-readable medium suitable for storing electronic instructions (e.g., computer programming code, such as software or firmware).

Various methods described herein may be practiced by combining one or more machine-readable storage media containing the code according to the present invention with appropriate standard computer hardware to execute the code contained therein. An apparatus for practicing various embodiments of the present invention may involve one or more computers (or one or more processors within a single computer) and storage systems containing or having network access to computer program(s) coded in accordance with various methods described herein, and the method steps of the invention could be accomplished by modules, routines, subroutines, or subparts of a computer program product.

If the specification states a component or feature “may”, “can”, “could”, or “might” be included or have a characteristic, that particular component or feature is not required to be included or have the characteristic.

Although the present disclosure has been described with the purpose of using a wearable device such as a watch, band, or like device, for monitoring health conditions and controlling home automation networks, it should be appreciated that the same has been done merely to illustrate exemplary embodiments and any other purpose or function for which the explained structure or configuration can be used, is covered within the scope of the present disclosure.

Exemplary embodiments will now be described more fully hereinafter with reference to the accompanying drawings, in which exemplary embodiments are shown. This invention may, however, be embodied in many different forms and should not be construed as limited to the embodiments set forth herein. These embodiments are provided so that this disclosure will be thorough and complete and will fully convey the scope of the invention to those of ordinary skill in the art. Moreover, all statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

Thus, for example, it will be appreciated by those of ordinary skill in the art that the diagrams, schematics, illustrations, and the like represent conceptual views or processes illustrating systems and methods embodying this invention. The functions of the various elements shown in the figures may be provided through the use of dedicated hardware as well as hardware capable of executing associated software. Similarly, any switches shown in the figures are conceptual only. Their function may be carried out through the operation of program logic, through dedicated logic, through the interaction of program control and dedicated logic, or even manually, the particular technique being selectable by the entity implementing this invention. Those of ordinary skill in the art further understand that the exemplary hardware, software, processes, methods, and/or operating systems described herein are for illustrative purposes and, thus, are not intended to be limited to any particular named.

Exemplary embodiments of the present disclosure are directed towards a multi-purpose wearable portable device that can be configured to collect/compile/collate/process data from one or more sensors and compare the data with one or more predefined threshold values stored in a memory or database to suggest appropriate actions and/or generate alert messages and communicate such messages/signals/communications to the wearer of the portable device and/or to a remote device and/or to remote healthcare system and/or to a home automation control system or to any other stakeholder/caretaker/doctor, among other entities or a combination thereof, all of which are within the scope of the present disclosure.

An aspect of the proposed wearable device can enable a user of the device to send emergency messages through one or a combination of emails, SMS's, MMS's, calls, push notifications or any other means to one or more stakeholders including but not limited to doctor, caretaker, family member, call center, hospital emergency, among other entities. Wearable portable device of the present disclosure can further enable tracking of user's location to help stakeholders to identify and locate precise position of the user whenever needed. In another aspect, healthcare data of a patient, monitored by means of one or more sensors can also be uploaded on a remote server/database so as to enable one or more people to view the user's profile, say on a dashboard, which can help them evaluate current health status, progress/decline in various configurable parameters in the last few days/months/years. With the device being portable, such health-related data can be uploaded by the device automatically or periodically or based on user intervention, or based on occurrence of certain actions, in real-time. Device of the present disclosure can further be configured to enable the user/caretaker to set one or more reminders to enable the user to take medications, perform exercises, or execute any other desired action timely.

In another aspect, an objective of the present disclosure is to provide a multi-purpose wearable device that can collect sensory data related to personal health and/or home automation control system, process the data based on a predefined set of rules/algorithms/thresholds, communicate alert message(s), any of which may be stored in a memory or database, to a wearer of the device and/or to a remote healthcare provider and/or automatically control the home automation control devices such as door lock, air conditioner, humidifier etc.

In yet another aspect, user wearable device such as a smart watch or a belt or glass or head strip of the present disclosure can include a programmable microcontroller operatively coupled with one or more embedded sensors or one or more sensors externally connected to the device through wireless communication means, a processing means to process data collected from one or more sensors, an alert/message generation means to generate alerts/messages for the wearer or remote device, and a communication means for transferring generated alerts/message(s) to one or more remote devices using a long range communication means. The one or more sensors, attached to the microcontroller of the wearable device, can include but are not limited to a heart-beat sensor, a pulse rate sensor, an accelerometer, a gyroscope, an oxygen sensor, a temperature sensor, a humidity sensor, a location tracking sensor, a UV sensor, a magnetometer, a proximity sensor, among other like sensors that can be individually and collectively be configured to collect one or more health parameter and enable control of the home automation network through desired parameters.

Another object of the present disclosure is to provide a wearable device having a configurable one touch emergency notification bottom to notify emergency team/healthcare provider through a communication means and/or unlock the door of house/building for emergency healthcare team/health care providers. This will help the emergency rescue crew to act quickly without losing time breaking the door.

Another object of the present disclosure is to provide an automatic system configured in a wearable device to issue command(s)/signal(s) to emergency team/healthcare provider and/or to home automation control system in case it detects an unwanted condition based on reading of one or more sensors that are coupled with the device. Based on the received command, home automation system can unlock door of house/building, adjust temperature/humidity/oxygen concentration within house/building, along with performing other actions, either pre-configured or configured on demand that are to be implemented.

Yet another object of the present disclosure is to provide a user wearable portable device that performs sleep apnea test with the help of data readings from an accelerometer and a gyroscope, which can be configured in or operatively coupled with the wearable device. The sleep test, in another implementation, can further be conducted based on oxygen saturation readings from either an in-built sensor or by means of a short range wireless communication of the device with an external pulse oximeter. Results of the test can then be shared with stakeholders including the user, doctor, caretaker, among other entities.

Still another object of the present disclosure is to provide a user wearable portable device that includes an embedded or operatively coupled GPS sensor(s) and/or triangulation based location estimation system(s), which can inform a healthcare provider about actual location of the user in case of an emergency.

Still another object of the present disclosure is to provide a wearable portable device that can provide suggestions/alert to a user based on reading(s) extracted from one or more sensors, and/or adjust setting of home automation control device. For example, user wearable device of the present disclosure can retrieve temperature and humidity data from applicable sensors, and suggest the concerned user about precautions to be taken at a given temperature/humidity, and/or if authorized, automatically adjust thermostat and/or humidifier to bring temperature and humidity at the required/desired/intended level.

Another object of the present disclosure is to provide a wearable portable device having embedded long term wireless communication means and/or short range wireless and/or a USB interface to receive and transmit sensory data and other messages between user wearable device and a remote health care provide and/or a home automation control system.

Further object of the present disclosure is to automatically post the reading/suggestions/alert messages on a user's registered social media websites, and receive suggestions from his/her friend using a user wearable portable device. In another embodiment, any other mode of message communication such as through a phone call, SMS, MMS, email, among others can be incorporated.

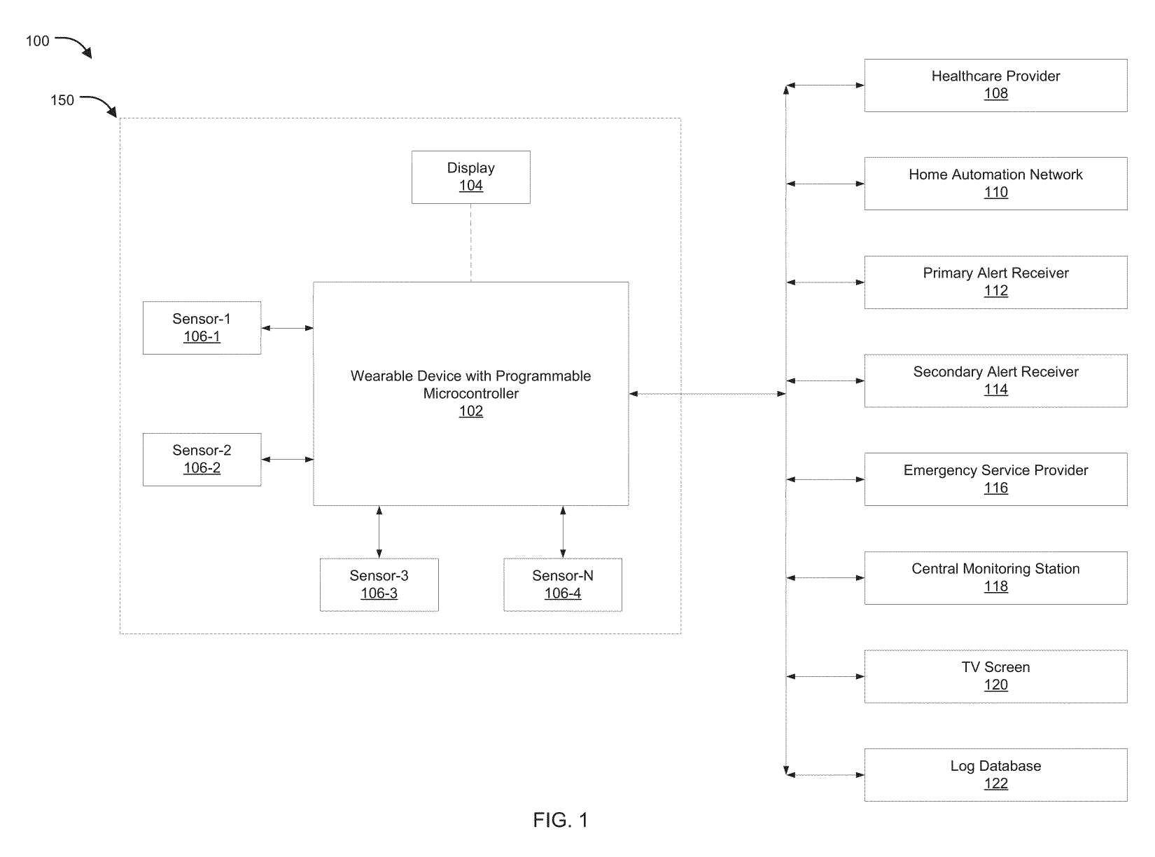

FIG. 1 illustrates an exemplary architecture diagram 100 showing a user wearable portable device 150 used for healthcare monitoring and home automation control in accordance with an embodiment. In an embodiment of the present disclosure, the user wearable portable device 150 can include a programmable microcontroller 102, one or more sensors 106-1, 106-2, 106-3, . . . , 106-n, collectively referred to as sensor(s) 106 hereinafter, which can either be embedded within the device 150 or can be external to the device 150 and operatively coupled thereto, and may further be operated by microcontroller 102. Device 150 can further include an optional display screen 104 to enable user to configure options, settings, and define/amend/modify/activate/deactivate parameters and controls. One should appreciate that although the device 150 is shown with respect with having one or more sensors, display, and a microcontroller, any other element/component or type therefore such as touch screen display, speaker, antenna, MIC, among others can be configured and all such elements/components are within the scope of the present disclosure. Any suitable processor such as an ARM based processor can be incorporated in an aspect. Additionally, any desired memory or database capabilities may be associated with device 150 and accessible or controllable by microcontroller 102. The device can take any size/shape and can be configured in any form factor including but not limited to a watch, band, necklace, garment, and the like. Although the present disclosure has, at certain instance, been described with respect to a watch, also referred to as a smart watch, the same is not limiting in any manner whatsoever, and any form factor is within the scope of the present disclosure.

In an embodiment, one or more sensors 106 can be embedded within the user wearable portable device 150 or can be operatively/externally coupled to it through a short range communication means such as WIFI, ZigBee®, and Bluetooth®, among other like protocols. Sensors 106 can include, but are not limited to, one or a combination of a heart-beat sensor, a pulse rate sensor, an accelerometer, a gyroscope, an oxygen sensor, a temperature sensor, a humidity sensor, a location tracking sensor, a UV sensor, a magnetometer, a proximity sensor, among other sensors based on the need, such that the sensors, independently or collectively, collect health parameters and also enable control of the home automation network.

In another embodiment, microcontroller 102 can include a memory unit (not shown) for storing a pre-loaded program, along with storing and enabling processing of data collected from one or more sensors 106. The programmable microcontroller 104 can be configured to process collected data (stored in the microcontroller memory or in any other external/operatively coupled memory) based on a preloaded method/algorithm to either format the data, process the data or to generate one or more alerts/messages/suggestions for user and/or remote healthcare provider 108. In an embodiment, the healthcare provider 108 can be configured as a remote system that can log/store/present healthcare parameter outputs received from the device 150 in say a central server. Such presentation can be made on a dashboard or any other appropriate/desired user interface on a Computer/Mobile/Tablet like devices to enable one or more stakeholders such as doctors, nurses, caretakers, family members, among other authorized people to view and give instructions to the device 150 to perform one or more actions. Therefore, the wearable device 150 can be configured such that it can receive and/or transmit parameters by means of messages/signals, which can take any format/means. In another embodiment, device 150 can also be configured to enable information/data of healthcare parameters monitored from a user of the device to be sent to one or more users, get posted in real-time on Applications/websites/databases such as on social networking websites, wherein the type of information and the manner in which the same is presented can be configured by the user by means of the settings/options configured within the device 150.

According to another embodiment, wearable device 150 can further be configured to control home automation network 110 by means of signals being sent through one or more sensors 106 that the device 150 is coupled with. For instance, in case an emergency is detected, device 150 can, based on user preferences, issue control signals to the home automation network 110 to unlock the door to enable emergency rescue. Such control signals can either be issued automatically by detection of certain configured events such as significant change in health parameters, or can be issued by means of an option being pressed/chosen by the user on the device 150, which activates the command for unlocking of the door. Likewise, any other aspect of the home automation network 110 such as controlling of air conditioners, heaters, refrigerators, television, among other like resources, are completely within the scope of the present disclosure and can be controlled/monitored/set by a single click of the device 150 or can be done automatically based on one or more pre-defined events or events which can be programmed in real-time.

In another embodiment of the present disclosure, device 150 can be operatively coupled with a primary alert receiver 112 and a secondary alert receiver 114, wherein such receivers can either be entities such as hospitals, call centers, emergency centers, or can include one or more professionals/people who the user wishes to be notified based on outcome of one or more healthcare parameters. Conditions based on which one or more receivers need to be notified can be defined/customized/configured by the user of the device 150 or can be set as default. Furthermore, primary receiver 112 can, for instance, include the caretaker of the user/patient and/or can include the doctor. Similarly, the secondary receiver can include family members, friends, among other stakeholders in the healthcare process. Communication to such receivers and remote servers/systems, for presentation/storage and/or sending alerts/messages can be performed by means known in the art such as through SMS, MMS, phone call, push notifications or other formats defined by the device 150 in context. For example, when the device detects that the heart-beat and/or blood pressure has gone beyond a defined threshold value stored in a memory, an alert can directly be sent in a defined format by the device 150 to the concerned/configured stakeholders. At the same time, multiple thresholds can be monitored/compared with, such that, for instance, in case a given health parameter goes beyond a first threshold value stored in a memory, only the secondary receivers can be informed, wherein case the same parameters exceeds a second threshold, the primary receivers can be notified. The device can also dynamically change the receivers based on one or more factors such as location of the user, previous history/track record, alert conditions set by the user, time of the day, among other like factors.

In continuation of the above, device 150 can also be operatively coupled with an emergency service provider 116 that can be sent one or more alerts/messages by the user based on critical health parameters. One should appreciate that such providers can also be combined with primary receivers or can be configurable separately, all of which are within the scope of the present disclosure. Emergency service providers 116 can also change based on user's location, nearest hospital, insurance policy, time/day, among other like parameters.

In another embodiment, device 150 can be operatively coupled with a central monitoring station 118, which can be a remote system and/or a remote server and can be configured to interact, in real-time, with the device 150 to control healthcare parameters, issue commands/instructions for user to perform, present data to stakeholders, send reminders to the user for taking medicines, perform physical exercises, along with undertaking other desired/planned/proposed actions. In an exemplary implementation, device 150 such as a smart watch can interact with devices like Bluetooth®/ZigBee® certified medical devices or home bases sensors and transfer readings to a central station/server 118, at which station 118 healthcare parameters from sensors/devices operatively coupled with the device 150 such as EKG, pulse oximeter, blood pressure sensor, body scale sensor, chest belt, motion sensor, windows sensor, door lock sensor among others can be evaluated and assessed to issue instructions back to the user to perform actions along with giving potential reasons for such parameters and steps that should be taken to rectify/correct/stabilize the condition.

In another embodiment, device 150 can be operatively coupled with a local and/or a remotely configured TV 120 or set top box like devices so that health parameters, past history, medical condition, among any other user/device related detail can be presented. Such a TV station 120 can be touch enabled to improve the efficiency of navigation and help the stakeholders such as caretakers and/or doctors to view desired details, zoom/magnify images, along with processing any other information that may be desired. In another exemplary embodiment, data collected from one or more sensors 106 of the device 150 can be sent to a log database such as a remote server 122 at regular intervals, or in real-time for archiving the data, enabling efficient processing of the data, which can be used for analysis and getting a trend of user's health condition over a period of time.

As explained above, in an example implementation, user wearable portable device 150 can be a multipurpose device that can collect/process data using one or more configured sensors to compare the same with one or more predefined threshold values and based on the same, suggest one or more actions and/or generate/communicate alerts/suggestions/messages to the wearer and/or to a remote device and/or health care system and/or to home automation control system. In an example implementation, user wearable portable device 150 can be configured for specific use such as for health care monitoring only or only for home automation control and can be configured to multiple purposes.

According to one embodiment, the device 150 can have a two-way call feature and also include a one touch emergency button to connect to say an emergency receiver, say 911, or to a call center during emergency. Device 150 of the present disclosure, an another embodiment, can further include a location tracking means such as a GPS or means to use cell tower triangulation to help locate the precise position of the user in context and issue instructions from the remote monitoring station to guide user to take one or more actions.

According to another embodiment, medicine or task reminders can also be given to the user by means of displaying the same on device display 104. Such reminders can be configured such that they need to be acknowledged by the user/wearer, which acknowledgement can be logged by the device 150 or in the log database 122. In an implementation, in case the wearer does not acknowledge the reminder, an alert can be sent to a caregiver.

System of the present disclosure can also be provided by means of a portal or an online system that can provide each user with a username and password, wherein after successful authentication, the user or other authorized set of stakeholders can view all current and previously stored healthcare data/conditions. All instructions, reminders, daily routine exercises, food to be taken, medicines to be consumed, among all other conceivable parameters can be defined by the doctor so that the user can view the same at any time. Various preferences such as inactivity, recursive motion, gesture, etc., can also be set on the portal or any other system application that is implemented in accordance with an embodiment of the present disclosure. All these parameters/preferences/rules/algorithms can be configurable by the user and can be downloaded by the wearable device and program the microcontroller either periodically or a specific request to program on demand by either a push message from web server, or SMS from web server or user, or in response to a HTTP/HTTPS post data request from the wearable device to portal or user action on the wearable device.

In yet another embodiment, device 150 of the present disclosure can include an accelerometer to determine fall of the wearer, wherein, in implementation, during a fall, an automatic alert can be sent to caregiver, For Example, an epilepsy patient wearing the watch gets a sudden attack of seizures, device can automatically detect based on the data from sensors like accelerometer, gyroscope, magnetometer, ambient light sensor, gesture detection like repeated shaking, current heart rate etc. and an alert can be sent to care givers. In another example, a senior citizen falling down in snow or stumbling a block an alert can be sent.

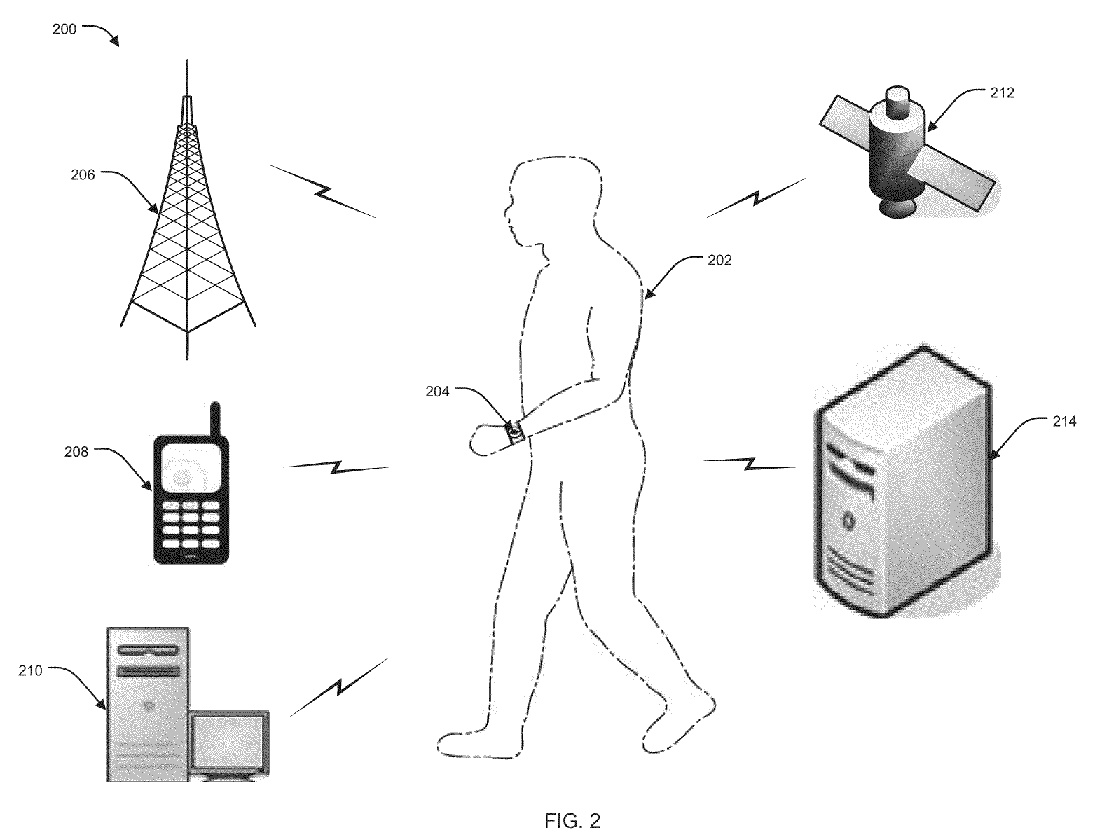

FIG. 2 illustrates an exemplary schematic view of an application environment 200 of user wearable portable device in accordance with an embodiment of the present disclosure. User wearable portable device 204 can be a multipurpose device that collects, processes, and monitors health care data of a user of the device, and can also enable the user to control the home automation network automatically or based on user commands. According to an example implementation, user wearable portable device 204 can be a smart watch, interchangeably referred to as watch 204 hereinafter. In an implementation, care receiver 202, also interchangeably referred to as patient or user 202 hereinafter, can wear the watch 204 all the time without holding it, or placing the device in the pocket of worn garments or place it in belt clip or wear as pendant. In an example environment 200, a care receiver 202 can wear a watch 204 that can be configured to collect data/health parameters from one or more sensors incorporated therein or operatively coupled thereto, and provide raw data and/or messages/alerts/communications based one or more protocols/formats to one or more devices such that to a local device 210, a cellular phone 208, a cellular radio station 206, a satellite 212, and remote server/station 214, among other conceivable devices such as remote systems, a web server, a web portal, log databases, and the like.

According to one embodiment, device 204 can also be configured to receive, through a receiver configured in the device 204 or through sensors operative to receive commands/instructions, one or more signals/communications from external devices/systems/databases in order to enable the user to perform or enable performance of the received communications. For instance, a doctor can, through a portal, send instructions to the patient/user 202 to perform certain exercises or take certain medications after defined time intervals, wherein such instructions can then be received by the device 204 from the portal or any other communication means, and be configured to give reminders/alerts to the user to perform the defined/desired actions.

In an embodiment, watch 204 can be provided with one or a combination of a programmable microcontroller, a display means, one or more sensing means, a positioning means, a Bluetooth® communication means, an emergency help calling means, a wireless communication means, a wireless transmission means, and a memory unit. Watch 204 can be bi-directionally connected with one or a combination of the local device 210, the cellular phone 208, the cellular radio station 206, the satellite 212, and the remote server 214, such that the watch/device 204 can receive messages/alerts/instructions/communications/signals from any of the devices, display it on the watch screen or use its speaker to audibly pronounce the instructions and/or alert the care receiver that there is a new message. Any other means to get the received message across to the intended recipient including the user of the device is completely within the scope of the present disclosure. Watch 204 can also be configured to receive GPS location from satellite 212 or determine its location based on triangulation method.

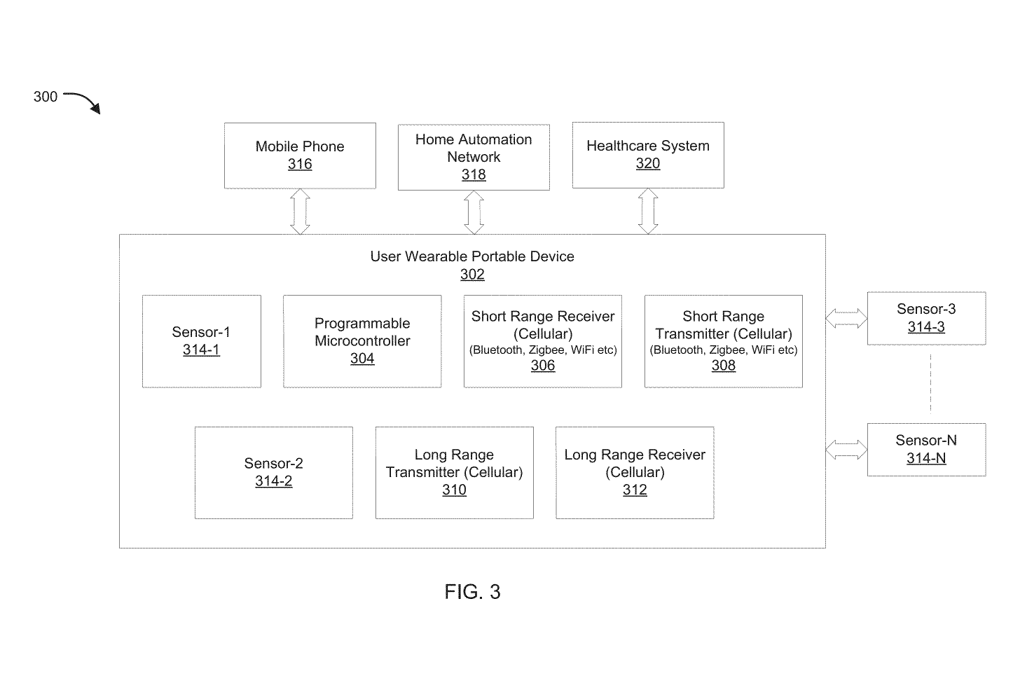

FIG. 3 illustrates an exemplary block diagram 300 of a user wearable portable device having long range and short range communication capability in accordance with an embodiment. As illustrated, user wearable portable device 302 can include a programmable microcontroller 304, a short range receiver 306, a short range transmitter 308, a long range transmitter 310, a long range receiver 312, a plurality of embedded sensors such as sensor-1 314-1, sensor-2 314-2, and plurality of communicatively coupled sensors such as sensor-3 314-3 to sensor-N 314-N, collectively referred to as sensors 314 hereinafter.

According to one embodiment, the programmable microcontroller 304 can include a processing unit, a storage unit, and other computing resources configured to receive one or more instructions over a short range receiver 306 and/or a long range receiver 312. Data received by programmable microcontroller 302 over short range receiver 306 can include data collected from one or more embedded sensors or communicatively coupled sensors. According to one embodiment, it is possible for user wearable device 302 of the present disclosure to receive sensory data from home automation controller sensors such as sensor-3, 314-3 that is not embedded within the wearable device 302. In another embodiment, it is possible for the wearable device 302 of present disclosure to receiver heart-beat signal from a separate heart-beat sensor installed in the user premises such as sensor-N through short range receiver 306 of the user wearable device. Short range receiver 306 can include a WI-FI receiver, a ZigBee® receiver, a NFC receiver, and a Bluetooth® receiver, or a combination thereof and can also include any other receiver configured for short range wireless communication.

In another embodiment, wearable portable device 302 of the present disclosure can have a long range communication receiver 312 and a long range transmitter 312 for communication with devices such as mobile phone 316, home automation control network 318, and health care systems 320, which can be remotely located from the wearable device 302. In an example implementation, long range receiver 312 and long range transmitter 310 can be used to directly receive and send data and/or message and/or alert and/or control signal from/to the home automation network 318 and/or the health care system 320. In an exemplary implementation, one or both of the home automation network 318 and the healthcare system 320 can be configured within the home/building in which the user of the device 302 resides, in which case, short range communication means can also be used for transmitting data to, and receiving commands/data/instructions from, the systems. Any other construction and/or mode of communication with external devices such as remote servers, log databases are completely within the scope of exemplary embodiments described herein. Furthermore, transceivers can also be configured in the device 302 to enable a single component to enable the purpose of both transmitting as well as received information/data.

According to one embodiment, device 302 can further include an in-built GPS that can be programmed to send current location of the user at regular intervals. In an implementation, when the GPS signal strength is weak, the proposed system that operatively works with the wearable device can enable the device to predict the current location based on cell tower triangulation method. In another embodiment, device 302 can have a geo-fence feature, which enables a geographical boundary to be set for each user/patient such that when the patient goes outside the boundary, an alert is set and communicated to one or more stakeholders.

In an example implementation, device 302 of the present disclosure can be synchronized with one or more mobile phone(s) 316 such that health parameters extracted by means of one or more sensors coupled with the device 302 can be presented on the mobile phone display. Mobile phones 316 can be operated by the user, caretaker, doctor, family member(s) or any other desired stakeholders who would like to monitor, receive health parameter data, and issue instructions/reminders/commands to the user to perform one or more actions.

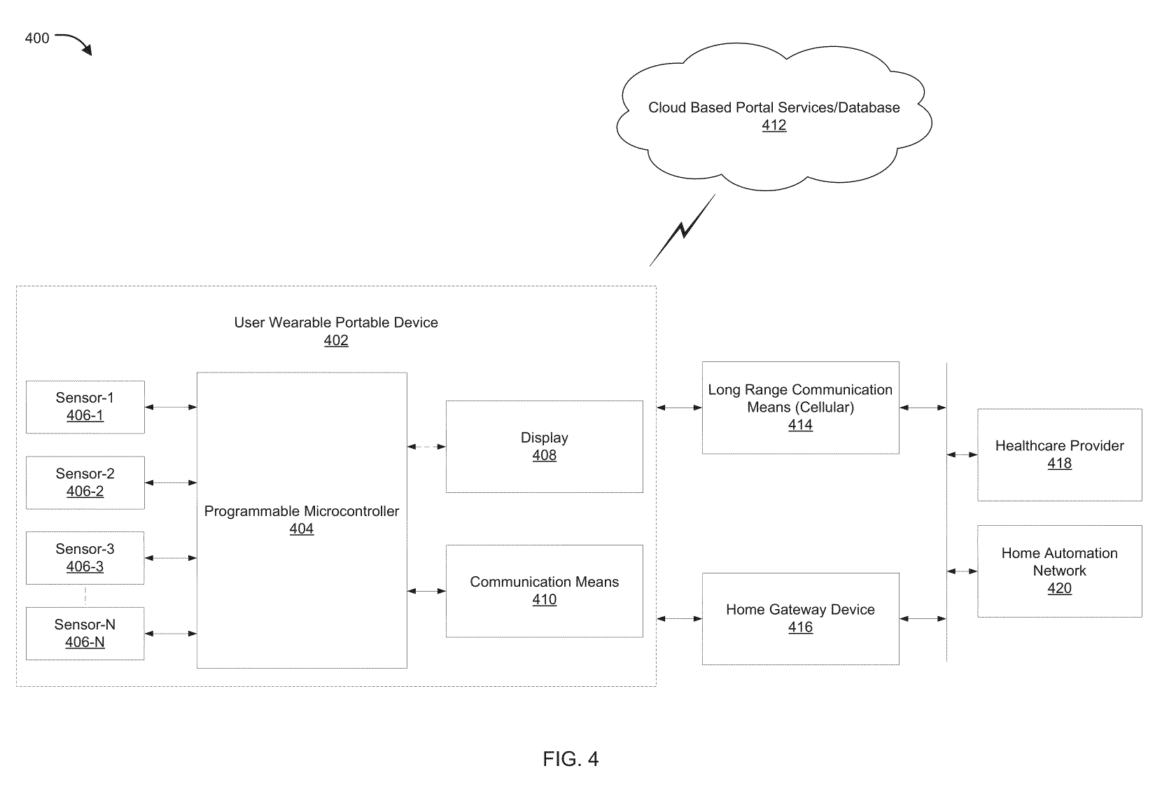

FIG. 4 illustrates an exemplary block diagram 400 of a remote healthcare system 418 and home automation control system 420 using a user wearable portable device 402 in accordance with an embodiment of the present disclosure. As illustrated, the user wearable portable device 402 can include an embedded programmable microcontroller 404, one or more sensors 406-1, 406-2, 406-3, . . . , 406-N, collectively referred to as sensors 406 hereinafter, a display 408, and a communication mean 410 such as short range communication means (Bluetooth®, ZigBee®, WIFI), and long range communication means such as through cellular communication.

In an embodiment, one or more sensors 406 can be configured to monitor/collect/retrieve/extract healthcare data of a user of the device 402 and send/transmit such raw data or processed data to the remote healthcare system 418 by means of long range communication means such as cellular communication. System 418 can receive and process the information/data and perform one or more desired actions including but not limited to alerting the user to perform one or more activities, giving progress report, issuing medications to be taken, presenting past history, projecting recovery plan, among undertaking other actions, all of which are within the scope of the present disclosure.

In another embodiment, device 402 of the present disclosure can send instructions to control the home automation network 420 based on detection of one or more events. For instance, in case of a heart attack or in case a user suddenly faints, condition of the user can be assessed/evaluated/detected and lock of the door can automatically be opened. Such actions can also take place based on explicit instructions from the user of the device 420. Conditions on which defined events need to take place can be set by the user, or can be set as default, or can be defined by any other stakeholder in accordance with an embodiment. Such conditions can be set either in a remote system or on a portal or within the device 402 of the present disclosure. Conditions can always be customized and modified as desired by the user or other authorized set of stakeholders. In an implementation, home automation network parameters can be extracted into the device 402 and made configurable such that based on conditions assessed by the device 402 such as temperature, humidity, or other health parameters, actions on the home resources/equipment's can be performed. For instance, using appropriate sensors, temperature and humidity of the surroundings from the device 402 can be calculated, and user of the device can be guided about precautions to be taken and if the user approves, the device 402 can, through communication means, modify the settings of thermostat or humidifier, which is either directly or indirectly communicatively coupled with the device 402.

In an example implementation, long range communication means 414 and home gateway device 416 can receive data from an embedded communication means such as short range communication means 410 of the user wearable portable device 402. In a specific example, long range communication means 414 and home gateway device 416 can receive data from an embedded short range communication means 410 of the user wearable portable device 402 using a WIFI or ZigBee® or Bluetooth® or NFC interface. A home gateway device 416 of the present example can be an integrated home automation and health care system. In an example implementation, the user wearable portable device 402 can send data to a cloud based portal or database 408 for other applications and data analysis. Such portal, as mentioned above, can be configured to present a dashboard showing, for each user of device 402, health parameters and information including but not limited to the past and current health condition trends, progress or decline in health condition, medications to be taken, reminders to be issued, among other desired information. The portal can be hosted on a remote system/server and can store all or part of the data generated by the device 402 such that data of interest from a physiological or biological or therapeutic perspective can be presented and view can be modified by each stakeholder based on the type/category of data that the user wishes to view.

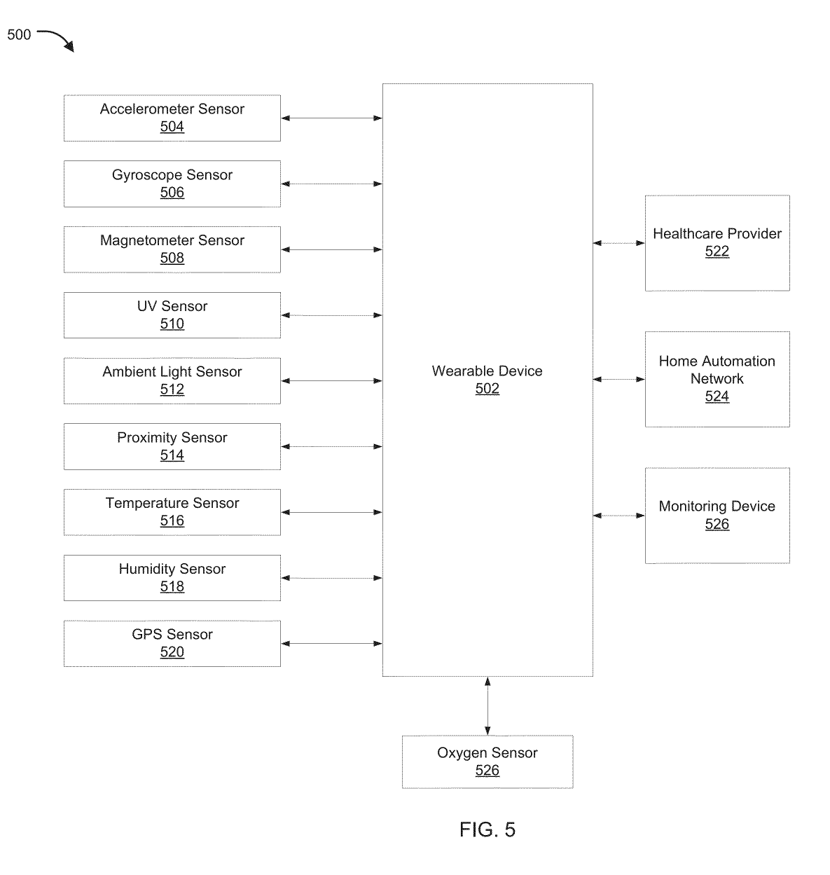

FIG. 5 illustrates an exemplary block diagram 500 of exemplary sensors embedded and/or attached to the user wearable portable device 502 that can be used by remote health care system and home automation control system in accordance with an embodiment of the present disclosure. As explained in earlier sections, sensors can either be configured within the user wearable portable device 502 or can be external to the device 502 and operatively coupled thereto. Device 502 can use data collected/determined/detected by these sensors for different applications, objectives, which can include, but are not limited to, monitoring health conditions of users, detecting environment characteristics such as temperature/humidity/sun intensity, identifying location, among other like objectives. Data from multiple sensors can also be processed together to determine certain health parameters and enhance the reliability of the decision.

In an example implementation, user wearable portable device 502 can include or be operatively coupled with an accelerometer 504 for functions like to detect and relay fall or turn off the backlight of LCD at certain conditions, a gyroscope 506 to detect and relay oscillations, and a magnetometer sensor 508 to detect and relay the nearing magnetic field and alert the wearer about that if he is wearing a pacemaker, an Oxygen sensor 526 which is configured to read oxygen saturation and use data from these sensors to conduct sleep apnea test. Results from the test can also be sent to an external health care system such as to a healthcare system 522 and/or to a remote server/system. In another example implementation, user wearable portable device 502 can use the accelerometer 504 data to determine sudden fall of the wearer, which can enable the device 502 to automatically send an automatic alert indicating a fall to caregivers.

In an example implementation, once the device 502 detects an emergency situation, it can be configured to send an emergency alert message(s) to health care system 522 and/or to home automation network 524 and/or to a specific home automation control device such as a door, and instruct the device to act in a defined manner. In a specific example, if a user of the device 502 detects an emergency condition, the device 502 can notify an ambulance service, a doctor, a primary health care provider (nurse), and also send a command to unlock door of house/building, which can help the emergency rescue crew to act quickly without losing time in breaking the door.

In an example implementation, device 502 can be configured to use and collect data from a temperature sensor 516 and a humidity sensor 518 and provide a guide/suggestion to the wearer/user about precautions to be taken and/or if authorized, modify settings of thermostat or humidifier to bring the temperate and humidity within the optimal range.

In another example implementation, user wearable portable device 502 can be configured to collect data from one or a combination of a GPS sensor (to get location, speed and altitude data), a UV sensor 510, a temperature sensor 516, and a humidity sensor 518 and provide a suggestion to wearer about water intake or SPF they should be using. User wearable portable device 502 can use the formula cited as below to calculate the SPF and warn the wearer if the exposure time exceeds the threshold time.

Minutes to Burn Without Sun Screen*SPF number=Maximum Sun Exposure Time

In an example implementation, for example, if the altitude is greater than 8,200 feet (2,500 meters), and speed calculated from GPS data is 2.5 miles or more and temperature is above 40° C., the device 502 can suggest how much water the wearer/user should take. For example, if the user/wearer is planning to work out in gym for one and half hours in the environment cited above (i.e. temperature 40° C., altitude 2,500 meters, running at the rate of 2.5 miles per hour), the device 502 can suggest the user to drink about 16 ounces (500 mL) of cool or cold water 1 to 2 hours before exercise, and drink about 16 ounces (500 mL) of cool water or a sports drink 15 minutes before the exercise. User wearable device can also suggest and remind the wearer to drink about 5 ounces (150 mL) of cool water every 10 minutes during exercise, and drink about 16 ounces (500 mL) of cool or cold water or a sports drink just after exercise. Other data from sensors such ambient light sensor 512 which can be programmed to read and relay heart rate, oxygen saturation in the body, proximity sensor 514 which can be programmed to detect and relay if the wearable device is on users body or measure the distance, magnetometer sensors 520, other sensors whose application is described above can be used for one or more different applications. For instance, a magnetometer 520 can warn people nearby magnetic fields, especially for people having pacemakers. Similarly, light sensor 512 can be used to determine light and automatically turn on/off the back light of the LCD or any other display characteristics. Proximity sensor 514, on the other hand, can be used to identify if the device 502 is on the user's hand or is removed. The sensor can also measure the heart rate and pulse oximetry.

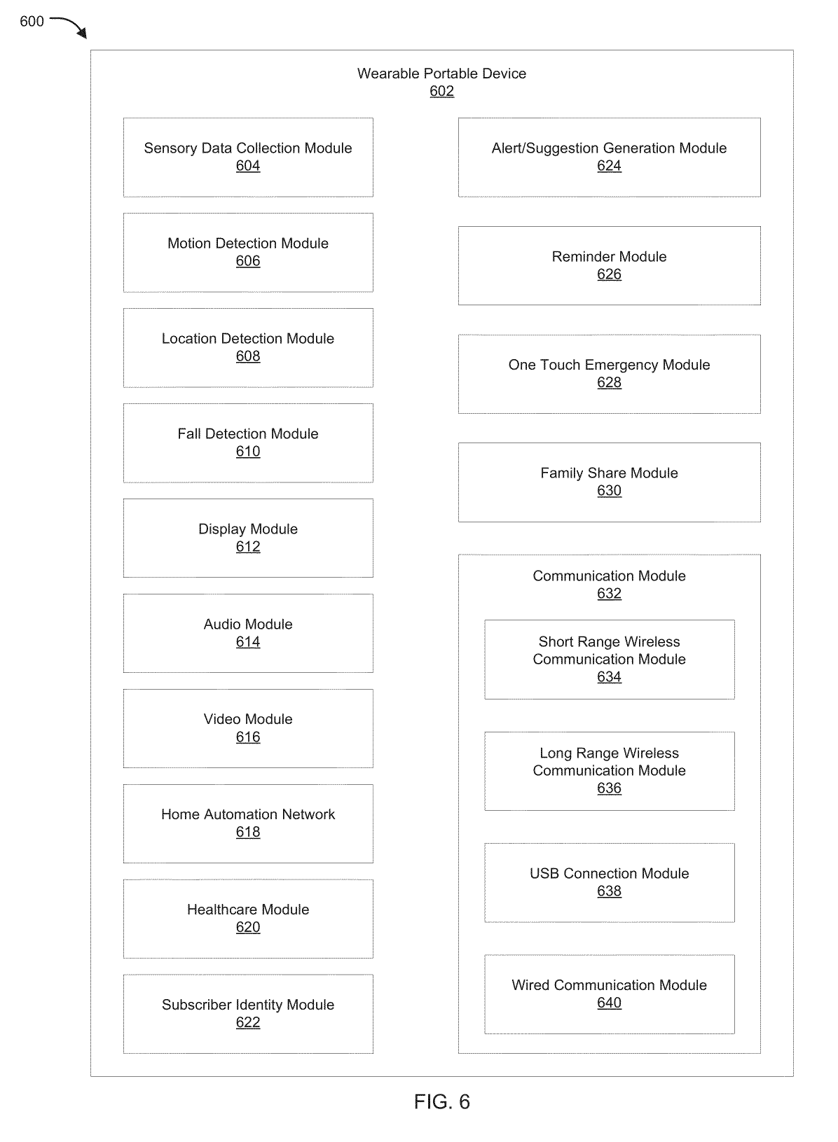

FIG. 6 illustrates exemplary functional modules 600 of a multipurpose user wearable portable device 602 used for remote health care management and home automation control in accordance with an embodiment. In an exemplary implementation, multipurpose wearable portable device 602 can include one or a combination of multiple functional modules, which can be incorporated in or coupled with a wearable device 602 based on user's configuration and customization. The modules/functions can also be enabled/disabled. In another embodiment, the modules can be combined to form a joint-module or can be further divided into sub-modules as desired and configured. In another embodiment, although the modules are shown to be functionalized/implemented within the device 602, one or more of the functional modules can be configured outside the system/device 602.

In an embodiment, device 602 can include a sensory data collection module 604, a motion detection module 606, a location detection module 608, a fall detection module 610, a display module 612, an audio module 614, a video module 616, a home automation control module 618, a health care system module 620, a subscriber identity module 622, an alert/suggestion generation module 624, a reminder module 626, a one touch emergency module 628, a family share module 830, and a communication module 632. For the convenience of reading and writing, multipurpose user wearable portable device 602 can be referred hereafter as wearable device 602 hereinafter.

In another embodiment, communication module 632 can further include a short range wireless communication module 634, a long range wireless communication module 636, USB connection module 638, and a wired communication module 640.

In an exemplary implementation, sensory data collection module 604 can be configured to periodically or randomly or in real-time collect data/information from one or more embedded sensors or communicatively coupled sensors including but not limited to a heart-beat sensor, a pulse rate sensor, an accelerometer, a gyroscope, an oxygen sensor, a temperature sensor, a humidity sensor, a location tracking sensor, a UV sensor, a magnetometer, a proximity sensor, an ambient light sensor, among other sensors used for health care management and home automation systems. Data/information collected by the module 604 can be processed, either by the device, or by any other local or remote processing system to obtain meaningful information/alerts/messages based on which actions can be expected to be done by the user and/or by the home automation network and/or by healthcare provider and/or caretaker and/or any other stakeholder.

In another embodiment, motion detection module 606 can be configured to detect movement of the user/wearer having the device and compare the movement with one or more defined conditions as to whether the moment is acceptable and within desired range, based on which alerts/messages can be generated. Location detection module 608 can be configured to determine location of the wearer based on data obtained from the GPS or based on triangulation method. Location determination module 608 can also be configured to enable determination of the altitude, latitude, and longitude of the wearer at regular intervals. In an implementation, using data collected by sensory data collection module 604 and location determination module 608, motion detection module 608 can determine speed of the wearer by which he is moving from one location to another location.

In an example implementation, fall detection module 610 can be configured to detect a sudden fall of a user using an accelerometer and using the data obtained from the accelerometer to determine the reason for fall and send alerts to one or a combination of caretaker, healthcare provider, doctor, among other people responsible. Data can also be collected by the sensory data collection module 604 and stored in a remote server or any other local or in-build memory.

In another embodiment, the user wearable device 602 can be configured to conduct a sleep apnea test using the accelerometer, a gyroscope, and oxygen saturation readings from either an in-built sensor of the device 602 or communicatively coupled through short-range wireless to an external pulse oximeter. In an implementation, if, based on the oxygen saturation readings, the device 602 detects depletion in oxygen concentration and alert the wearer, the results can be provided to the healthcare system and necessary alert/messages can be implemented to take control of the situation.

In an example implementation, display module 612 of the multipurpose user wearable device 602 can be configured to display data collected from sensory data collection module 604 directly on display screen or can display one or more suggestions/messages/alerts as generated by other modules. Similarly, audio module 614 of the device 602 can be configured to read out data as collected from the sensors. In a similar implementation, animations and/or active graphs can be created by video module 616 based on the data collected by sensory data collection module 604 through use of one or a combination of sensors and can be displayed on screen of the wearable device 602. Data collected by sensors from the device 602 can also be presented on a remote display device or on a portal/web interface or a system/software interface to enable one or more stakeholders to view/filter/process the report.

The wearable device 602 can also include a home automation control module 618 configured to use data of one or more sensors collected by sensory data collection module 602 and analyse working status of the home automation control devices such as thermostats, humidifiers, oxygen sensors, ambient lights, secure system etc. in order to analyse and/or issue one or more control commands to these home automation control devices over a short range communication medium. In an example implementation, home automation control module 618 can be configured to issue a command to an automatic door, which, upon receiving a valid command, can open the door of house/building. For example, if the wearable device 602 determines, by means of the fall detection module 610, that a user has fallen, control module 618 of the present disclosure can be configured to issue instructions to automatically issue one or more commands to open the door so that emergency team does not need to struggle to open the door. In another example, home automation control module can issue a command to a thermostat to increase/decrease room temperature and bring to optimal level.

In an exemplary implementation, wearable device 602 can have a health care module 620 configured to use healthcare data collected from one or more sensors to determine health status of the wearer based on one or more preloaded algorithm(s) and threshold value(s). Healthcare module 620 of wearable device 602 can further be configured to detect emergency health conditions of the wearer if value(s) of the one or more parameters collected from one or more sensors exceeds a predefined value. For example, if the heart-beat sensor reads heart-beat of 90 bpm (beat per minute), it can automatically issue a health emergency signal to a health care provider such as local nurse, or to a primary health care provider, or to a secondary health care provider. On serious cases, it can automatically send a request to emergency team such an ambulance service or doctor. While sending the emergency message, the health care module 620 can also send location data collected from location determination module 608 to the emergency service provider, which can help the provider to reach the user quickly. In such emergency scenario, the health care module 620 can invoke a message to home automation control module 618 to issue a door unlock command so as to quickly provide access of the wearer to the emergency team.

Health care module 620 and home automation control module 618 can be configured to use an alert/suggestion generation module 624 to provide one or more alerts/messages in one or more formats to the wearer indicating the condition of the user along with giving instructions/procedures/reminders among other indicators. The alert/suggestion generation module 624 of the wearable device 602 can also be configured to suggest the wearer as to how to escape from building in emergency condition, how much water he/she should take in the given temperature, exercises to be done, medication to be taken, among like suggestions. Device 602 of the present disclosure can further include a subscriber identify module 622 configured to authenticate/authorize user of the device. For instance, the device can be configured with an authentication means such as username/password, eye scan, biometric means, voice biometric information, facial biometric information, fingerprint scan, among other known schemes to authenticate and ensure that the user wearing the device 602 is authorized to do so. Such a module 622 can be initialized sooner the device is picked up or worn by the user such that the device can authenticate the user through one or a combination of known authentication means and then enable the device for access by the user.

In an example implementation, wearable device 602 can include a reminder module 626 that provide a reminder or alert message to a wearer to complete an activity such as to take his/her medicine on time, visit a doctor on schedule time and date, purchase his/her medicine before it is over, among other like activities. A wearer can also set out reminder such as to his visit his friend/family, complete an office job, call a friend, among other like reminders.

In an example implementation, apart from the automatic messages generated by home automation control module 618 and health care module 620, wearer device 602 can further be configured to provide a one touch emergency module 628 that can provide an option to the wearer to manually indicate an emergency situation. Wearer device 602 can include an interface such as a button that can be pressed/touched by a wearer to indicate an emergency condition. On a single touch/press of that button, the one touch emergency module 628 can send a command to one or more healthcare provider(s), caretakers, among other stakeholders along with issuing desired/configured control signals to the home automation control device.

In an example implementation, wearer device 602 can include a family share module 630 configured to enable sharing of raw and/or processed healthcare data along with reminders/messages/alerts/communications to one or more family members/friends. Such data can be shared periodically or intermittently or randomly. Family share module 630 can also be configured to provide and share sensor data or processed data directly on social media platform. It can post a message to registered friends or family about status such health status, current location, among other information of the wearer including, for instance, how much distance he/she has covered in last 1 hour, how many calories has the wearer burnt, among other like parameters. On emergency cases such as a medical emergency, family share module 630 of wearer device 602 can also be configured to call family on one or more registered number(s), or send his family a text message on registered number.

In an example implementation, wearer device 602 can be configured to send or receive data/messages/alerts/commands from care taker nurse, doctors, family or friends or emergency response teams using communication module 632 that can include a short range wireless communication module 634, a long range wireless communication module 636, a USB connection module 638, and a wired communication module 640. Short range wireless communication module 634 can be configured to send/receive data using Bluetooth® or ZigBee® or WIFI or NFC or any other wireless communication technique used for short range data communication. Long range wireless communication module 636, on the other hand, can be configured to send/receive data using cellular network techniques such as Wide Area Network, GSM, CDMA or new wireless communication means such 2G, 3G and 4G. In an example implementation, if the wearable device 602 does not include a long range wireless communication module 638, it can send the data using short range wireless communication module 636 to a mobile phone or a home gateway that can further send the data/messages/alerts/commands to the remote devices/servers.

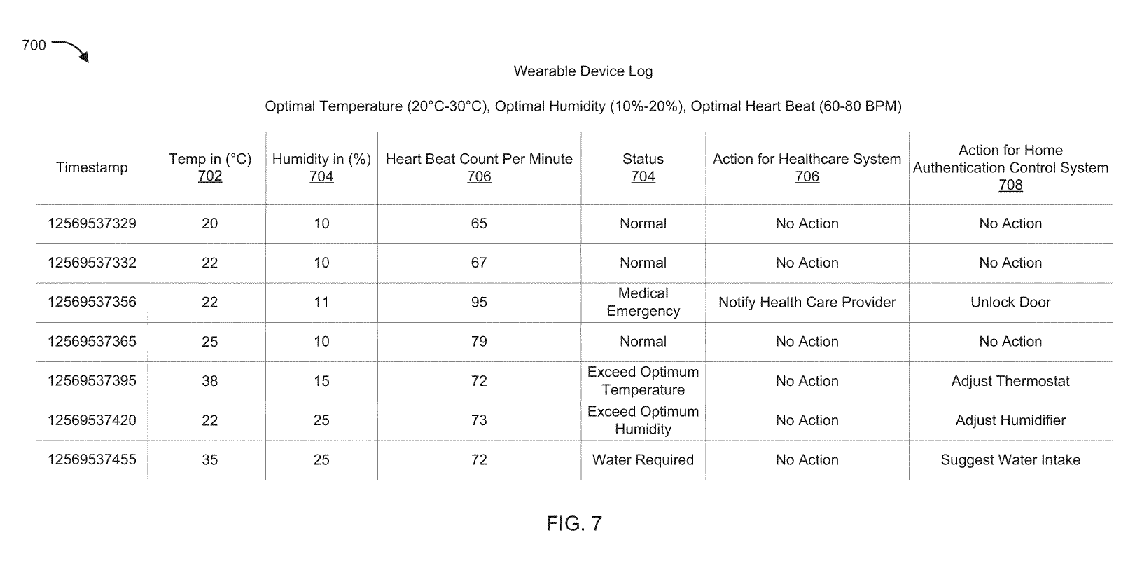

FIG. 7 illustrates an exemplary logical table 700 showing actions taken by proposed wearable device based on readings received from one or more sensors in accordance with an embodiment. One should appreciate that table 700 is completely exemplary and logical in nature and has only been presented demonstrate an exemplary embodiment and such a table may not necessarily exist in physical memory/space. At the same time, such a table 700 can also be incorporated for logging in different times and actions that were taken on/by healthcare system and taken on/by the home automation network. Any number of parameters can be incorporated in the table and any number of actions can be undertaken including but not limited to, issuing multiple alerts, suggestions, messages, not only to the user in context, but also to the remote system, server, web portal, system application, along with other stakeholders such as doctors, caretakers, among others. The exemplary table 700 shows data collected from three sensors namely temperature sensor, humidity sensor, and heart-beat sensor stored as temperature in (° C.) 702, humidity in (%) 704, and heart beat count per minute 706 in different columns of the table 700. Based on the readings from the sensors, wearable device can determine health/home automation control status 704 and take one or more actions for health care system 706 and for home automation network 708. Proposed wearable device of the present disclosure can be configured to take one or more actions based on a pre-defined set of rules/methods/algorithms. In an example, wearable device can be configured to take an action if temperature exceeds a predefined or goes below an optimal temperature range such as 20° C. to 30° C., or in case humidity exceeds/goes below a predefined optimal humidity range such as 20%-30%, and in case heart-beat exceeds/goes below a predefined optimal heart-beat rate such as 60-80 bpm.

In reference to table 700, it can be seen that the table includes entries for various timestamps, wherein status is normal for all timestamps where the values of the parameters are within threshold ranges. However, for timestamp 12569537356, as the heart beat goes above the threshold, the status 704 is declared as emergency and the healthcare provider can be notified and the home door can be automatically unlocked. One should appreciate that such a representation is very simplistic and there can be many threshold values, different stakeholders to be informed at different times, and therefore many more configurations and possibilities can be incorporated to make the system comprehensive and reliable. Similarly, for timestamp 12569537395, as the temperature is above defined threshold, the status 704 can be declared as exceeding temperature and accordingly the thermostat can be adjusted.

While embodiments of the present invention have been illustrated and described, it will be clear that the invention is not limited to these embodiments only. Numerous modifications, changes, variations, substitutions, and equivalents will be apparent to those skilled in the art, without departing from the spirit and scope of the invention, as described in the claim.

What is claimed is:

1. A wearable portable communication device comprising:
a wearable device;
a microcontroller;
a plurality of sensors operatively coupled to said microcontroller to sense one or more health parameters based on conditions determined by the microcontroller;
a pre-loaded program executed by the microcontroller for storing and enabling processing of data collected from one or more of said plurality of sensors; and
a display on the wearable device that displays one or a combination of health parameters, alerts, messages, and reminders to a user wearing said wearable device, wherein said wearable device is operatively coupled by the microcontroller and a transmitter communicatively coupled with a home automation network and configured to control said home automation network and perform at least one of specific actions on said one or more sensory data and specific user actions that are either pre-programmed or programmed on demand.

2. The device of claim 1, wherein said one or more health parameters are heart rate, blood pressure, breath rate, body temperature, oxygen level, sudden fall, location, physiological parameters, psychological parameters, and biological parameters.

3. The device of claim 1, wherein said plurality of sensors are an accelerometer, gyroscope, magnetometer, UV sensor, light sensor, proximity sensor, location sensor, temperature sensor, humidity sensor.

4. The device of claim 1, wherein said wearable device is one of a watch, a belt, spectacles, a band, and a head strip.

5. The device of claim 1, wherein said location sensor is one of a GPS locator and triangulation method-based location identification means.

6. The device of claim 1, wherein said wearable device further comprises a first home automation control option, wherein selection of said first home automation control option enables unlocking of door of home of said user.

7. A wearable portable communication device comprising:
a microcontroller housed in a wearable communication device;
a plurality of sensors operatively coupled to, and controlled by, said microcontroller and configured to sense one or more surrounding parameters, said surrounding parameters selected from a group comprising temperature, humidity, location, magnetic field, light, and object proximity;
a pre-loaded program executed by the microcontroller for storing and processing data collected from one or more of said plurality of sensors; and
a display on the wearable communication device to display one or more of a combination of health parameters, alerts, messages, and reminders to a user wearing said device, wherein said device is operatively coupled by a transmitter on the wearable communication device with a home automation network and configured to control said home automation network based on said one or more surrounding parameters.

8. The device of claim 7, wherein said plurality of sensors comprise at least one of an accelerometer, gyroscope, magnetometer, UV sensor, light sensor, proximity sensor, location sensor, temperature sensor, humidity sensor.

9. The device of claim 7, wherein said device is one of a watch, a belt, spectacles, a band, and a head strip.

10. The device of claim 7, wherein a change in one or more of said one or more surrounding parameters is compared, by the microcontroller, with respective threshold values stored in a database associated with the microcontroller to control said home automation network.

11. The device of claim 10, wherein a detection of temperature by one of the plurality of sensors that is beyond a defined temperature threshold range stored in a database associated with the microcontroller triggers the microcontroller in said device to issue control signal by a transmitter for changing thermostat settings of said home automation network.

12. The device of claim 10, wherein a detection of humidity by one of the plurality of sensors that is beyond a defined humidity threshold range stored in a database associated with the microcontroller triggers the microcontroller in said device to issue control signal by a transmitter for changing humidifier settings of said home automation network.

13. A wearable portable communication device comprising:
a microcontroller;
a plurality of sensors operatively coupled to said microcontroller and configured to sense one or more health parameters;
a pre-loaded program stored in a memory and executed by the microcontroller for storing and enabling processing of data collected from one or more of said plurality of sensors; and
a display configured to display one or more of health parameters, alerts, messages, and reminders to a user wearing said device, wherein said device is operatively coupled by a transmitter with a remote healthcare system and is configured to transmit and receive one or more of messages, alerts, instructions, communications to or from said remote healthcare system, wherein said remote healthcare system enables monitoring of said one or more health parameters.

14. The device of claim 13, wherein said microcontroller of said device is operatively coupled with a database configured to log at least one part of said one or more health parameters.

15. The device of claim 13, wherein said one or more health parameters comprise heart rate, blood pressure, breath rate, body temperature, oxygen level, sudden fall, location, physiological parameters, psychological parameters, and biological parameters.

16. The device of claim 13, wherein said plurality of sensors comprise an accelerometer, gyroscope, magnetometer, UV sensor, light sensor, proximity sensor, location sensor, temperature sensor, and humidity sensor.

17. The device of claim 13, said device further comprising a selectable emergency option, wherein selection of said emergency option enables a user of said device to transmit one or more alerts to a defined number of stakeholders, wherein said stakeholders are selected from at least one of a doctor, healthcare provider, hospital, nurse, family member, caretaker, and a health-concerning stakeholder.

18. The device of claim 13, further comprising:
a geo-fence in the device configured to mark a logical geographical boundary for said user, and use a location sensor to determine an exact location of said user, the device further raising at least one of an alert, a message, a communication, and a notification to at least one stakeholder when said user moves out of said logical geographical boundary.

19. The device of claim 13, wherein said device is configured with an accelerometer for detecting a sudden action of said user, and said device issues one or more of an alert, a message, a communication, and a notification to at least one stakeholder.

20. The device of claim 13, wherein said device is configured to at least receive or generate one or more reminders to take medicines, perform physical actions, and perform one or more pre-defined actions.

21. The device of claim 20, wherein, if said user does not acknowledge said one or more reminders, one or more of an alert, a message, a communication, and a notification is sent to at least one stakeholder.

22. The device of claim 13, wherein said device is operatively coupled with a software application configured to present at least one part of said one or more health parameters.

23. The device of claim 22, wherein said device is a web-based application.

24. The device of claim 13, wherein said device measures, with an operatively coupled pulse oximeter, oxygen saturation readings to enable performance of sleep apnea test.

25. The device of claim 1, wherein the microcontroller of the wearable device is configured to perform at least one of specific action on said one or more sensory data from at least one sensor that is either built-in the device or commutatively coupled and specific user actions that are either pre-programmed or programmed on demand

26. The device of claim 1, wherein the wearable device is communicatively coupled with a webserver where user preferences and programming instructions are stored to load on demand.

27. The device of claim 26, wherein the webserver provides access to user of all the sensory data, alerts, reminders, preferences and actions to be performed.
